(19) United States
(12) Patent Application Publication
(10) Pub. No.: US 2023/0147505 A1
(43) Pub. Date: May 11, 2023

(54) IDENTIFYING POOR CARDIORESPIRATORY FITNESS USING SENSORS OF WEARABLE DEVICES

(71) Applicant: Apple Inc., Cupertino, CA (US)

(72) Inventors: Katherine Niehaus, San Francisco, CA (US); Britni A. Crocker, Santa Cruz, CA (US); Maxsim L. Gibiansky, Sunnyvale, CA (US); William R. Powers, III, San Francisco, CA (US); Allison L. Gilmore, Redwood City, CA (US); Asif Khalak, Belmont, CA (US); Sheena Sharma, Carbondale, IL (US); Richard A. Fineman, Campbell, CA (US); Kyle A. Reed, Carmel, IN (US); Karthik Jayaraman Raghuram, Foster City, CA (US); Adeeti V. Ullal, Emerald Hills, CA (US)

(21) Appl. No.: 17/985,098

(22) Filed: Nov. 10, 2022

Related U.S. Application Data
(60) Provisional application No. 63/278,474, filed on Nov. 11, 2021.

(51) Int. Cl. A61B 5/11; A61B 5/00

(52) U.S. Cl. CPC A61B 5/1118; A61B 5/4866

(57) ABSTRACT

Embodiments are disclosed for identifying poor cardio metabolic health using sensors of wearable devices. In an embodiment, a method comprises: obtaining estimates of maximal oxygen consumption of a user during exercise; determining at least one confidence weight based on context data; adjusting the maximal oxygen consumption estimates using the at least one confidence weight; aggregating the adjusted maximal oxygen consumption estimates to generate a summary maximal oxygen consumption estimate and corresponding confidence interval for the user; and classifying cardiorespiratory fitness of the user based on at least one of the summary maximum consumption estimate, the corresponding confidence interval, a population error model or a low cardiorespiratory fitness threshold.

### CROSS-REFERENCE TO RELATED APPLICATION

This application claims priority to U.S. Provisional Patent Application No. 63/278,474, filed Nov. 11, 2021, the entire contents of which are incorporated herein by reference.

### TECHNICAL FIELD

This disclosure relates generally to fitness monitoring using sensors of wearable devices.

### BACKGROUND

Modern wearable devices (e.g., smart watches, fitness bands) are often used by individuals during fitness activities to determine their energy expenditure. Some wearable devices include inertial sensors (e.g., accelerometers, angular rate sensors) that are used to estimate a work rate (WR) based metabolic equivalent of task (MET) for the user wearing the device. Other sensors in the form of a global positioning system (GPS) and/or barometer may also contribute towards the WR estimate. Some wearable devices also include a heart rate (HR) sensor that provides HR data that can be used with the user's estimated maximal oxygen consumption (VO₂ max), which is the maximum amount of oxygen that can be consumed by the user during incremental exercise, and sometimes other data (e.g., user's weight, age) to estimate the user's HR based MET. The WR MET and HR MET are typically combined in some suitable manner (e.g., averaged) to determine the energy expenditure of the user.

### SUMMARY

Embodiments are disclosed for identifying poor cardiorespiratory fitness using sensors of wearable devices.

In an embodiment, a method comprises: obtaining estimates of maximal oxygen consumption of a user during exercise; determining at least one confidence weight based on context data; adjusting the maximal oxygen consumption estimates using the at least one confidence weight; aggregating the adjusted maximal oxygen consumption estimates to generate a summary maximal oxygen consumption estimate and corresponding confidence interval for the user; and classifying cardiorespiratory fitness of the user based on at least one of the summary maximal oxygen consumption estimate, the corresponding confidence interval, a population error model or a low cardiorespiratory fitness threshold.

In an embodiment, method further comprises: filtering the maximal oxygen estimates based on the context data to exclude estimates of maximal oxygen that do not indicate a low level of cardiorespiratory fitness of the user.

In an embodiment, the filtering further comprises: using the context data with a location of the user and time of day to identify transient inconsistencies in cardiovascular efficiency of the user that indicate low levels of cardiorespiratory fitness of the user; and excluding the estimates of maximal oxygen that do not indicate a low level of cardiorespiratory fitness of the user.

In an embodiment, determining the at least one confidence weight based on context data includes determining the at least one confidence weight based on environment context data.

In an embodiment, determining the at least one confidence weight based on context data includes determining the at least one confidence weight based on behavior context data.

In an embodiment, determining the at least one confidence weight based on context data includes determining the at least one confidence weight based on environment context data and behavior context data.

In an embodiment, the estimates of maximal oxygen consumption are generated based on mechanical work rate and heart rate energy expenditure models.

In an embodiment, a method comprises: determining, with at least one processor, confidence-weighted historical sensor observations and confidence-weighted maximal oxygen consumption estimates based on heart rate data, mechanical work rate data, input sensor quality, validity of a work rate model for providing the mechanical work rate data and a measure of observation consistency; determining, with the at least one processor, a first joint confidence based on the confidence-weighted historical sensor observations; determining, with the at least one processor, a second joint confidence based on the confidence-weighted maximal oxygen consumption estimates, historical maximal oxygen consumption estimates and context data; and determining, with the at least one processor, a cardiorespiratory fitness of the user by evaluating the first and second joint confidences using at least one criteria.

In an embodiment, the first joint confidence is generated by a first joint confidence model that includes a physiologic consistency model, a historical consistency model and an observation sufficiency model.

In an embodiment, the physiologic consistency model is configured to determine agreement between the confidence-weighted historical sensor observations and a physiologic model of how the normalized heart rate data responds to the mechanical work rate data.

In an embodiment, agreement is determined by an aggregate distance metric computed on the confidence-weighted historical sensor observations.

In an embodiment, the physiologic model is a classifier with an input feature vector that includes observation confidence weight, exertion level of the user and frequency of the observation.

In an embodiment, the historical consistency model is configured to determine whether there is agreement between the normalized heart rate data and the mechanical work rate data.

In an embodiment, wherein agreement is determined by an aggregate distance metric and a required consistency within a given exertion range.

In an embodiment, the observation sufficiency model is configured to determine whether there is a sufficient number of high confidence observations output by observation confidence model to determine the confidence weights.

In an embodiment, the observation sufficiency model is applied after passing physiologic and historical consistency thresholds.

In an embodiment, the observation sufficiency model is configured to determine whether there is a minimum exertion range of coverage or a requirement of a minimum number of observations across a minimum number of unique exercise periods or days.

In an embodiment, the second joint confidence is generated by a second joint confidence model that includes a personalized confidence interval model, an estimate sufficiency model, a personalized threshold model and an interpretability model.

In an embodiment, the personalized confidence interval is defined by an exponentially time and confidence-weighted average of the maximal oxygen consumption estimates, and a standard deviation of longitudinally-smoothed maximal oxygen estimates.

In an embodiment, the environment and behavior context data is processed by the adjustment model, which maps behavior or environment features of a given activity period to an adjusted maximal oxygen consumption estimate.

In an embodiment, the adjustment model includes a linear or non-linear model of the relationship between increased altitude, external or internal temperature, humidity, or other environmental data and a reduction in maximal oxygen consumption.

In an embodiment, the estimate sufficiency model determines if the maximal oxygen consumption estimates have converged and that there are sufficient number of estimates.

In an embodiment, the personalized threshold model determines a confidence interval around the maximal oxygen consumption estimate based upon a population error model of maximal oxygen consumption estimates, and whether the confidence interval is below a set threshold.

In an embodiment, the interpretability model ensures that a final classification result is reasonable and interpretable to the user by determining if a minimum number of recent maximal oxygen consumption estimates are below the set threshold.

In an embodiment, a criteria evaluator is used to determine if the confidence-weighted maximal oxygen consumption estimates meet specified criteria, and if so, notifying the user that they have persistently low cardiorespiratory fitness.

Other embodiments can include an apparatus, computing device and non-transitory, computer-readable storage medium.

Particular embodiments disclosed herein provide one or more of the following advantages. The low fitness identification processes described herein minimize the influence of temporary deviations in VO₂ max estimates using a weighted aggregation method and a robust evaluation method. The aggregation method produces a summary VO₂ max value and a personalized confidence interval on that value. These values are used together with a population-based confidence interval to evaluate the cardiorespiratory fitness of the user in comparison to a low fitness threshold.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 is a graph that illustrates short-term fluctuations versus persistently low cardiorespiratory fitness.

FIG. 2 is a block diagram illustrating a system for identifying persistently low cardiorespiratory fitness, according to an embodiment.

FIG. 3 is a block diagram further illustrating the system shown in FIG. 2 for identifying persistently low cardiorespiratory fitness, according to an embodiment.

FIG. 4 is a flow diagram of a process of identifying persistently low cardiorespiratory fitness, according to an embodiment.

FIG. 5 is example wearable device architecture for implementing the features and processes described in reference to FIGS. 1-4.

### DETAILED DESCRIPTION

### Overview

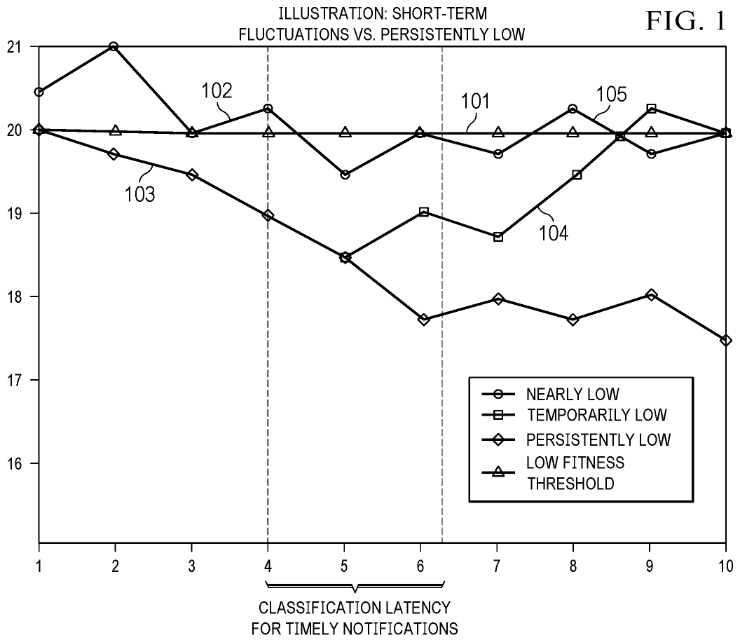

FIG. 1 illustrates short-term fluctuations versus persistently low cardiorespiratory fitness. Cardiorespiratory fitness metrics estimated from sensors on wearable devices can be used to identify people with poor cardiometabolic health, as determined by persistently low cardiorespiratory fitness, independent of short-term fluctuations in estimates due to user behavior and environmental factors.

A naive method for identifying individuals with low cardiorespiratory fitness (e.g., comparison of individual VO₂ _{ₘₐₓ} estimates to a fixed threshold) is subject to error from short-term fluctuations in the estimates made by wearable sensors. These fluctuations occur due to observability limitations (e.g., low wear time, scarce observations during intense physical activity), as well as behavioral factors and environmental factors that would typically be controlled in a clinical laboratory setting. For example, VO₂ max estimates acquired when the individual is carrying an unobserved load (e.g., a child or heavy backpack) or on more difficult terrain (e.g., sand) may differ from VO₂ max estimates acquired in ideal conditions. Environmental factors such as ambient temperature, humidity, and altitude may also cause differences in estimates.

Even using highly accurate methods to estimate VO₂ max from wearable sensors, occasional large deviations can be expected due to these challenges. For reference, gold standard laboratory exercise tests conducted with careful controls (e.g., fixed room temperature, requirement that patients fast before the test, etc.) exhibit variability of ˜5%. Reference devices that measure VO₂ max in more natural conditions, such as portable cardiopulmonary exercise testing systems exhibit variability of ˜10%.

Referring again to FIG. 1, the graph illustrates an example with a low fitness threshold 101, a nearly low cardiorespiratory fitness level trajectory 102, a persistently low cardio respiratory fitness level trajectory 103 and a temporarily low cardiorespiratory fitness level trajectory 104. The vertical axis represents VO₂ max and the horizontal axis represents units of time (e.g., days, weeks, months). Trajectory 104 includes a short-term dip in VO₂ max below threshold 101, followed by a recovery to a point above the low fitness threshold due to more accurate estimates. For users near the low fitness threshold 101, even small amounts of variation pose a challenge, as shown by trajectory 102. Thus, a method that naively looks for estimates below low fitness threshold 101 may falsely alert users, causing unnecessary alarm and, potentially, unnecessary medical care. Conversely, an overly cautious method may wait too long in pursuit of a stable measurement, missing the optimal window to help a user improve their health.

### Example System

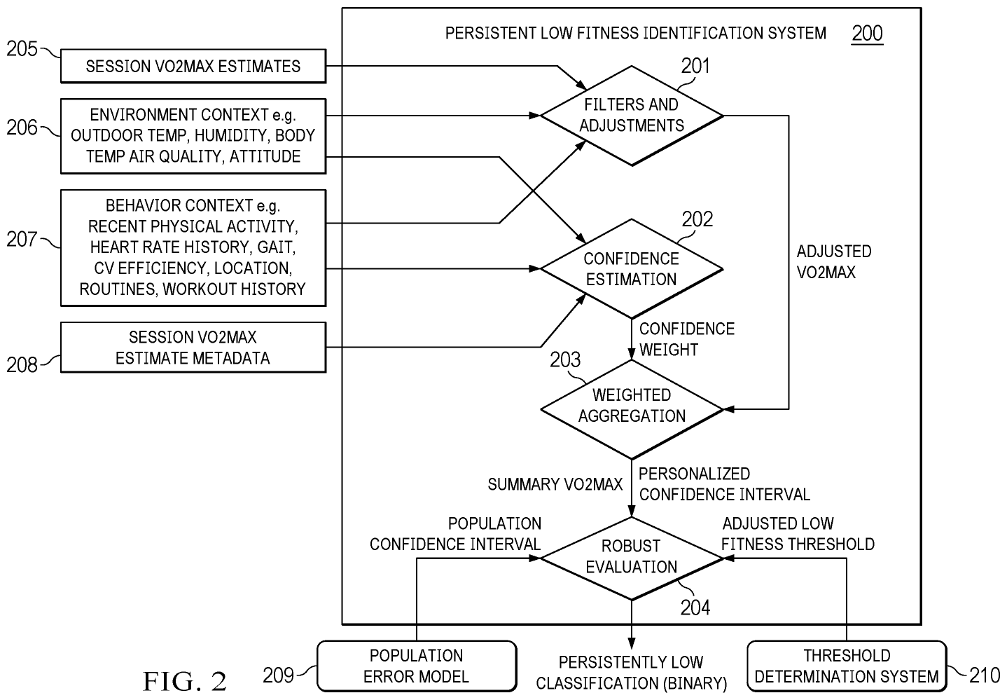

FIG. 2 is a block diagram illustrating system 200 for identifying persistently low cardiorespiratory fitness, according to an embodiment. System 200 includes VO₂ max estimate filter/adjuster 201, confidence estimator 202, weighted aggregator 203 and robust evaluator 204.

System 200 takes as input VO₂ max estimates, environment context data 206, behavior context data 207 and VO₂ max estimated metadata 208 (e.g., number of observations, number of days with observations, frequency of observations, duration of activity observed, range of exertion observed, maximum exertion observed, frequency of observations of exertion above threshold sensor measurement, confidence derived from population error models). In an embodiment, the VO₂ max estimates 205 are generated based on mechanical work rate and/or heart rate energy expenditure models. For example, a wearable device (e.g., a smartwatch, fitness band) can include a heart rate sensor that measures heart rate from which a maximum (HRₘₐₓ) and resting heart rate (HRᵣₑₛ) can be derived. VO₂ max can then be estimated using Equation [1]:

VO₂ Max˜15×(HRₘₐₓ/HRᵣₑₛ). [1]

Other examples of mechanical work rate and heart rate energy expenditure models are disclosed in, for example, U.S. Pat. No. 9,918,646.

Some examples of environment context data 206 include but are not limited to: outdoor temperature data, humidity data, body temperature data, air quality data, altitude data and the like.

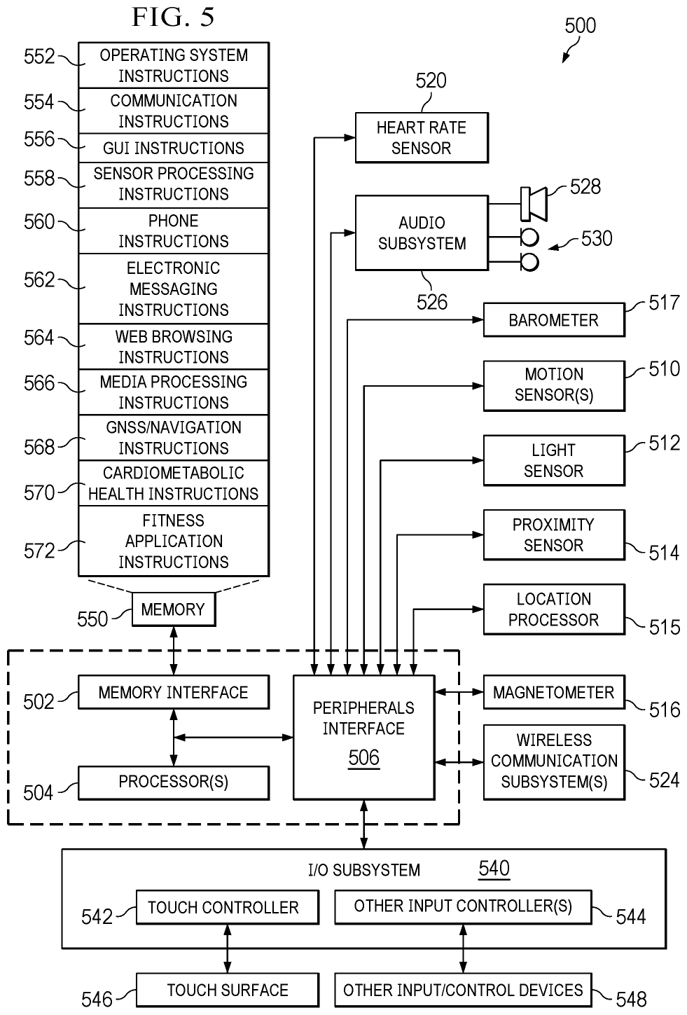

Some examples of behavior context data 207 include but are not limited to: recent physical activity, HR history, gait, cardiovascular efficiency, routines, workout history and the like. Some of the environment context data 206 and behavior context data 207 can be generated from sensors of the wearable device, such as ambient temperature sensors, humidity sensors, body temperature sensors, air quality sensors, altitude sensors (e.g., a barometric pressure sensor) and motion sensors (e.g., accelerometers, gyros), as described in reference to the device architecture 500 shown in FIG. 5.

Environment context data 206 and behavior context data 207 may also be used with location data (e.g., provided by a GPS receiver) and time of day to identify personal activity routines that can approximate more controlled behavioral and environmental conditions. Context data 206, 207 acquired during these periods can be used to identify transient inconsistencies in cardiovascular efficiency to identify true low levels of cardiorespiratory fitness.

System 200 uses environment context data 206 to identify VO₂ max estimates 205 obtained under confounding conditions that may cause an apparent acute drop in cardiovascular efficiency and therefore do not indicate a true low level of cardiorespiratory fitness. For example, ambient temperature data and/or location-based weather is used to adjust across a usual temperature range, filter extreme temperature values and determine a confidence weight. Humidity data is used to adjust VO₂ max estimates and determine a confidence weight. Altitude data is used to adjust VO₂ max estimates and determine a confidence weight. Terrain data is used to determine gait metrics, location and activity type (e.g., hiking) and to determine a confidence weight.

System 200 also uses behavior context data 206 to identify VO₂ max estimates 205 obtained under confounding conditions. For example, user fatigue can be determined based on recent physical activity detected by one or more motion sensors, historical HR observations stored on the wearable device, a measure of cardiovascular efficiency or user body temperature. Data indicative of user fatigue is used to filter and/or adjust VO₂ max estimates. Load data (e.g., the user is carrying a baby) can be determined from gait metrics (e.g., based on motion sensor data), a load classifier (e.g., a stroller classifier) or a measure of cardiovascular efficiency to filter and/or adjust VO₂ max estimates. Outlier user behavior can be identified by deviations from routine or typical activity patterns, reduction in recent sleep, increased respiration rate, decreased blood oxygen, elevated stress as assessed through heart rate variability (HRV) or other autonomic function. The outlier behavior can be used to filter and/or adjust VO₂ max estimates.

Context data 206, 207 described above can be derived from sensor data provided by a number of sensors embedded in or attached to the wearable device or by any other data source. Context data 206, 207 can also be derived by comparing “in-the-moment” observations to existing personalized physiological models. For example, in-the-moment observations of WR and HR relationship may be compared to an existing, personalized physiological model of the user's cardiorespiratory function to identify temporary deviations due to fatigue. These in-the-moment observations can be further confirmed using observations of recent user physical activity. For example, differences between the in-the-moment gait observations and an existing, personalized biomechanical model of the user's gait can be used to identify periods of activity on difficult terrain.

VO₂ max filter/adjuster 201 takes as input VO₂ max estimates 205, context data 206, 207 and filters the VO₂ max estimates 205 using context data 206, 207 to exclude VO₂ max estimates that that do not indicate a true low level of cardiorespiratory fitness due to confounding conditions. VO₂ max filter/adjuster 201 also adjusts the filtered estimates using a confidence weight and outputs the adjusted VO₂ max estimates to weighted aggregator 203. Weighted aggregator 203 aggregates the adjusted VO₂ max estimates into a summary VO₂ max estimate and a corresponding confidence interval that is personalized to the user. Context data 206, 207 is used by weighted aggregator 203 to generate the personalized confidence interval.

In an embodiment, the confidence weight is generated by confidence estimator 202 based on context data 206, 207 and session VO₂ max estimate metadata 208. The confidence weight indicates a confidence in the estimated VO₂ max estimates output by the WR and/or HR energy expenditure models.

In an embodiment, robust evaluator 204 uses two personalized confidence intervals to ensure that small changes in the user's summary VO₂ max value do not change the user's low cardiorespiratory fitness classification, even if the summary VO₂ max is very close to an adjusted low fitness threshold generated by threshold determination system 210. A first confidence interval is personalized based on a model that accounts for variability and trend in historical VO₂ max estimates 205 of the user, and adjusts for the confidence of those estimates. The second confidence interval is modeled using population error model 209, which is an empirical error distribution of VO₂ max estimates 205 in a reference population. These two personalized confidence intervals are implemented in robust evaluator 204 with tunable parameters that control a trade-off between robustness and sensitivity.

With optimal tuning, the weighted aggregation method implemented by system 200 is sensitive enough to identify individuals whose summary VO₂ max is only slightly below the low fitness threshold, but nonetheless robust to small fluctuations in summary VO₂ max over time. The weighted aggregation method also ensures that the total set of observations going into the evaluation of an individual's cardiorespiratory fitness is well-sampled in time and in exertion (mechanical work rate, heart rate). For example, while each individual VO₂ max estimate may be derived from a limited set of observations, the summary VO₂ estimate represents observations across an appropriately broad range. This is accomplished by combining the confidence weight produced with each VO₂ max estimate with VO₂ max estimated metadata 208. Examples of VO₂ max estimated metadata 208 include but are not limited to: number of observations, number of days with observations, recency of observations, duration of activity observed, range of exertion observed, maximum exertion observed, frequency of observations of exertion above a threshold and sensor measurement confidence derived from population error model 209. Requiring system 200 to accumulate a wide range of observations comes with a latency trade-off. If these requirements are too strong, there may be an unacceptable classification latency before a user can be identified as having persistently low cardiorespiratory fitness, or before the user's declining cardiorespiratory fitness is determined by system 200. In an embodiment, various parameters used by weighted aggregator 203 are tuned to optimize the personalized confidence interval versus latency trade-off.

Note that not all the components 201-210 of system 200 need to be included in a single embodiment. Other embodiments may use only a subset of the components 201-210.

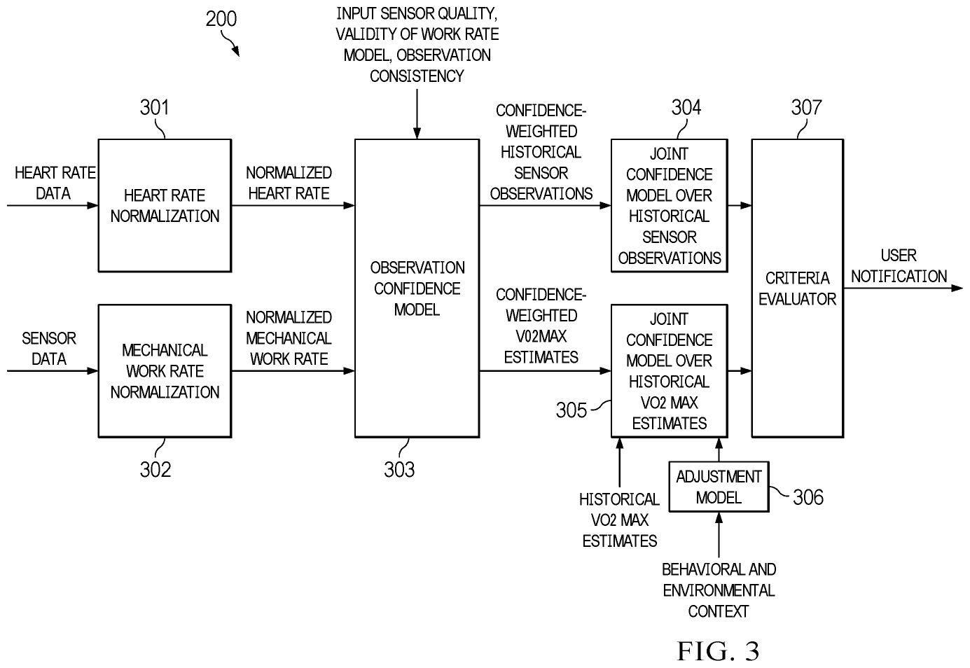

FIG. 3 is a block diagram further illustrating system 200 for identifying persistently low cardiorespiratory fitness, according to an embodiment. System 200 includes HR normalization module 301 for normalizing HR data and mechanical WR normalization module 302 for normalizing sensor data (e.g., acceleration, rotation rate, pressure, GPS data). The normalized HR data and normalized mechanical WR data are input into observation confidence model 303 which is configured to evaluate the quality of the observations and generate confidence-weighted HR and mechanical WR observation data.

In an embodiment, observation confidence model 303 can be a classification model trained to predict high or low quality observations. In an embodiment, observation confidence model 303 can be configured to provide a binary output or a continuous output of observation quality (e.g., a value between 0 and 1). In an embodiment, observation confidence model 303 implements one or more known classifying methods, including but not limited to: logistic regression, Naïve Bayes, random forests, neural networks and the like. In an embodiment, observation confidence model 303 uses one or more known regression tools, including but not limited to: linear regression, random forest regression and the like.

A first output of observation confidence model 303 are confidence-weighted sensor observations, which are input into joint confidence model 304. Joint confidence model 304 includes a physiologic consistency model, a historical consistency model and an observation sufficiency model. The physiologic consistency model is configured to determine agreement (i.e., consistency) between the confidence-weighted observations and a physiologic model of how normalized heart rate responds to normalized mechanical work-rate. In an embodiment, agreement is defined by an aggregate distance metric (e.g., Euclidean distance, KL divergence across a window) computed on the observations. In another embodiment, the physiologic model can be a classifier with an input feature vector that includes, for example, observation confidence weight, exertion level of the user and frequency of the observation.

The historical consistency model is configured to determine whether there is agreement between the normalized HR and the mechanical WR observations. In an embodiment, agreement can be defined by an aggregate distance metric (e.g., Euclidean distance) and a required consistency within a given exertion range.

The observation sufficiency model is configured to determine whether there is a sufficient number of high confidence observations output by observation confidence model 303 to determine the confidence weights. The observation sufficiency model can be applied after passing physiologic and historical consistency thresholds. In an embodiment, there is also a requirement of a minimum exertion range of coverage and/or a requirement of a minimum number of observations across a minimum number of unique exercise periods or days.

A second output of observation confidence model 303 are confidence-weighted VO₂ max estimates, which are input together with environment and behavior context data 206, 207 into joint confidence model 305. Joint confidence model 305 includes a personalized confidence interval model, an estimate sufficiency model, a personalized threshold model and an interpretability model. In an embodiment, a personalized confidence interval is defined by an exponentially time and confidence-weighted average of the VO₂ max estimates 205, and a standard deviation of longitudinally-smoothed VO₂ max estimates 205.

The environment and behavior context data 206, 207 is processed by adjustment model 306, which maps behavior or environment features of a given activity period to an adjusted VO₂ max estimate 205. In an embodiment, adjustment model 306 can include a linear or non-linear model of the relationship between increased altitude, external or internal temperature, humidity, etc., and a reduction in VO₂ max. The adjustment model 306 can be applied to an individual workout session.

The estimate sufficiency model determines if the VO₂ max estimates 205 have converged and that there are sufficient number of VO₂ max estimates 205. The personalized threshold model 210 determines a confidence interval around the VO₂ max estimate based upon a population error model 209 of VO₂ max estimates, and whether the confidence interval is below a set threshold determined by threshold determination system 210. The interpretability model ensures that the final classification result is reasonable and interpretable to the user, for instance by determining if a minimum number of recent estimates are below the set threshold.

Criteria evaluator 307 determines if the confidence-weighted VO₂ max estimates 205 meet all the criteria implemented by the models described above (e.g., passes all the thresholds). If so, the user is notified that they have persistently low cardiorespiratory fitness. The notification can be provided on a display of the wearable device or another device, through a message or push notification on the wearable device or other device, and/or an audio output and/or any other type of feedback and/or combination of feedback types.

### Example Process

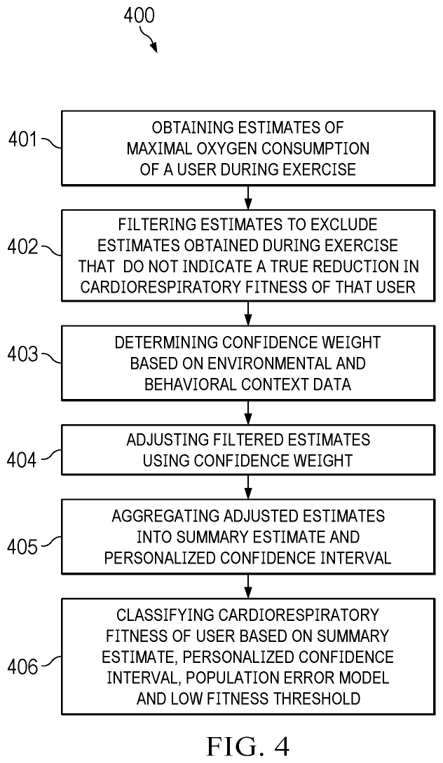

FIG. 4 is a flow diagram of a process 400 identifying poor cardiorespiratory fitness using sensors of a wearable device. Process 400 can be implemented using the wearable device architecture 500 disclosed in reference to FIG. 5.

Process 400 includes the steps of obtaining estimates of maximal oxygen consumption of a user during exercise (401); filtering the estimates based on environment context data and behavior context data to exclude estimates that do not indicate a true reduction in cardiorespiratory fitness of the user (402); determining at least one confidence weight based on the environment context data and the behavior context data (403); adjusting the filtered estimates using the at least one confidence weight (404); aggregating the adjusted estimates to generate a summary estimate and corresponding confidence interval for the user (405); and classifying cardiorespiratory fitness of the user based on the summary estimate, the corresponding confidence interval, a population error model and a low cardiorespiratory fitness threshold (406). Each of these steps were previously described in reference to FIGS. 2 and 3.

### Exemplary Wearable Device Architecture

FIG. 5 illustrates example wearable device architecture 500 implementing the features and operations described in reference to FIGS. 1-4. Architecture 500 can include memory interface 502, one or more hardware data processors, image processors and/or processors 504 and peripherals interface 506. Memory interface 502, one or more processors 504 and/or peripherals interface 506 can be separate components or can be integrated in one or more integrated circuits.

Sensors, devices and subsystems can be coupled to peripherals interface 506 to provide multiple functionalities. For example, one or more motion sensors 510, light sensor 512 and proximity sensor 514 can be coupled to peripherals interface 506 to facilitate motion sensing (e.g., acceleration, rotation rates), lighting and proximity functions of the wearable device. Location processor 515 can be connected to peripherals interface 506 to provide geo-positioning. In some implementations, location processor 515 can be a GNSS receiver, such as the Global Positioning System (GPS) receiver. Electronic magnetometer 516 (e.g., an integrated circuit chip) can also be connected to peripherals interface 506 to provide data that can be used to determine the direction of magnetic North. Electronic magnetometer 516 can provide data to an electronic compass application. Motion sensor(s) 510 can include one or more accelerometers and/or gyros configured to determine change of speed and direction of movement of the wearable device. Pressure sensor 517 (e.g., a barometer) can be configured to measure atmospheric pressure around the mobile device.

Heart rate monitoring subsystem 520 for measuring the heartbeat of a user who is wearing the device on their wrist. In an embodiment, subsystem 520 includes a PPG to detect a heartbeat.

Communication functions can be facilitated through wireless communication subsystems 524, which can include radio frequency (RF) receivers and transmitters (or transceivers) and/or optical (e.g., infrared) receivers and transmitters. The specific design and implementation of the communication subsystem 524 can depend on the communication network(s) over which a mobile device is intended to operate. For example, architecture 500 can include communication subsystems 524 designed to operate over a GSM network, a GPRS network, an EDGE network, a Wi-Fi™ network and a Bluetooth™ network. In particular, the wireless communication subsystems 524 can include hosting protocols, such that the mobile device can be configured as a base station for other wireless devices.

Audio subsystem 526 can be coupled to a speaker 528 and a microphone 530 to facilitate voice-enabled functions, such as voice recognition, voice replication, digital recording and telephony functions. Audio subsystem 526 can be configured to receive voice commands from the user.

I/O subsystem 540 can include touch surface controller 542 and/or other input controller(s) 544. Touch surface controller 542 can be coupled to a touch surface 546. Touch surface 546 and touch surface controller 542 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 546. Touch surface 546 can include, for example, a touch screen or the digital crown of a smart watch. I/O subsystem 540 can include a haptic engine or device for providing haptic feedback (e.g., vibration) in response to commands from processor 504. In an embodiment, touch surface 546 can be a pressure-sensitive surface.

Other input controller(s) 544 can be coupled to other input/control devices 548, such as one or more buttons, rocker switches, thumb-wheel, infrared port and USB port. The one or more buttons (not shown) can include an up/down button for volume control of speaker 528 and/or microphone 530. Touch surface 546 or other controllers 544 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 546; and a pressing of the button for a second duration that is longer than the first duration may turn power to the mobile device on or off. The user may be able to customize a functionality of one or more of the buttons. The touch surface 546 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 502 can be coupled to memory 550. Memory 550 can include high-speed random access memory and/or non-volatile memory, such as one or more magnetic disk storage devices, one or more optical storage devices and/or flash memory (e.g., NAND, NOR). Memory 550 can store operating system 552, such as the iOS operating system developed by Apple Inc. of Cupertino, Calif. Operating system 552 may include instructions for handling basic system services and for performing hardware dependent tasks. In some implementations, operating system 552 can include a kernel (e.g., UNIX kernel).

Memory 550 may also store communication instructions 554 to facilitate communicating with one or more additional devices, one or more computers and/or one or more servers, such as, for example, instructions for implementing a software stack for wired or wireless communications with other devices. Memory 550 may include graphical user interface instructions 556 to facilitate graphic user interface processing; sensor processing instructions 558 to facilitate sensor-related processing and functions; phone instructions 560 to facilitate phone-related processes and functions; electronic messaging instructions 562 to facilitate electronic-messaging related processes and functions; web browsing instructions 564 to facilitate web browsing-related processes and functions; media processing instructions 566 to facilitate media processing-related processes and functions; GNSS/Location instructions 568 to facilitate generic GNSS and location-related processes and instructions; and cardio metabolic health instructions 570 and fitness application instructions 572, as described in reference to FIGS. 1-4.

Each of the above identified instructions and applications can correspond to a set of instructions for performing one or more functions described above. These instructions need not be implemented as separate software programs, procedures, or modules. Memory 550 can include additional instructions or fewer instructions. Furthermore, various functions of the mobile device may be implemented in hardware and/or in software, including in one or more signal processing and/or application specific integrated circuits.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., SWIFT, Objective-C, C#, Java), including compiled or interpreted languages, and it can be deployed in any form, including as a stand-alone program or as a module, component, subroutine, a browser-based web application, or other unit suitable for use in a computing environment.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor, estimates of maximal oxygen consumption of a user during exercise;
determining, with the at least one processor, at least one confidence weight based on context data;
adjusting, with the at least one processor, the maximal oxygen consumption estimates using the at least one confidence weight;
aggregating, with the at least one processor, the adjusted maximal oxygen consumption estimates to generate a summary maximal oxygen consumption estimate and corresponding confidence interval for the user; and
classifying, with the at least one processor, cardiorespiratory fitness of the user based on at least one of the summary maximal oxygen consumption estimate, the corresponding confidence interval, a population error model or a low cardiorespiratory fitness threshold.

2. The method of claim 1, further comprising:
filtering, with the at least one processor, the maximal oxygen consumption estimates based on the context data to exclude estimates of maximal oxygen consumption that do not indicate a low level of cardiorespiratory fitness of the user.

3. The method of claim 2, wherein the filtering further comprises:
using the context data with a location of the user and time of day to identify transient inconsistencies in cardiovascular efficiency of the user that indicate low levels of cardiorespiratory fitness of the user; and
excluding the estimates of maximal oxygen consumption that do not indicate a low level of cardiorespiratory fitness of the user.

4. The method of claim 1, wherein determining the at least one confidence weight based on context data includes determining the at least one confidence weight based on environment context data.

5. The method of claim 1, wherein determining the at least one confidence weight based on context data includes determining the at least one confidence weight based on behavior context data.

6. The method of claim 1, wherein determining the at least one confidence weight based on context data includes determining the at least one confidence weight based on environment context data and behavior context data.

7. The method of claim 1, wherein the estimates of maximal oxygen consumption are generated based on mechanical work rate and heart rate energy expenditure models.

8. A method comprising:
determining, with at least one processor, confidence-weighted historical sensor observations and confidence-weighted maximal oxygen consumption estimates based on heart rate data, mechanical work rate data, input sensor quality, validity of a work rate model for generating the mechanical work rate data and a measure of observation consistency;
determining, with the at least one processor, a first joint confidence based on the confidence-weighted historical sensor observations;
determining, with the at least one processor, a second joint confidence based on the confidence-weighted maximal oxygen consumption estimates, historical maximal oxygen consumption estimates and context data; and
determining, with the at least one processor, a cardiorespiratory fitness of the user by evaluating the first and second joint confidences using at least one criteria.

9. The method of claim 8, wherein the first joint confidence is generated by a first joint confidence model that includes a physiologic consistency model, a historical consistency model and an observation sufficiency model.

10. The method of claim 9, wherein the physiologic consistency model is configured to determine agreement between the confidence-weighted historical sensor observations and a physiologic model of how the normalized heart rate data responds to the mechanical work rate data.

11. The method of claim 9, wherein agreement is determined by an aggregate distance metric computed on the confidence-weighted historical sensor observations.

12. The method of claim 9, the physiologic model is a classifier with an input feature vector that includes observation confidence weight, exertion level of the user and frequency of the observation.

13. The method of claim 9, wherein the historical consistency model is configured to determine whether there is agreement between a normalized heart rate data and a normalized mechanical work rate data.

14. The method of claim 12, wherein agreement is determined by an aggregate distance metric and a required consistency within a given exertion range.

15. The method of claim 9, wherein the observation sufficiency model is configured to determine whether there is a sufficient number of high confidence observations output by observation confidence model to determine the confidence weights.

16. The method of claim 9, wherein the observation sufficiency model is applied after passing physiologic and historical consistency thresholds.

17. The method of claim 9, wherein the observation sufficiency model is configured to determine whether there is a minimum exertion range of coverage or a requirement of a minimum number of observations across a minimum number of unique exercise periods or days.

18. The method of claim 9, wherein the second joint confidence is generated by a second joint confidence model that includes a personalized confidence interval model, an estimate sufficiency model, a personalized threshold model and an interpretability model.

19. The method of claim 18, wherein the personalized confidence interval is defined by an exponentially time and confidence-weighted average of the maximal oxygen consumption estimates, and a standard deviation of longitudinally-smoothed maximal oxygen estimates.

20. The method of claim 18, wherein the environment and behavior context data is processed by the adjustment model, which maps behavior or environment features of a given activity period to an adjusted maximal oxygen consumption estimate.

21. The method of claim 20, wherein the adjustment model includes a linear or non-linear model of the relationship between increased altitude, external or internal temperature, humidity, or other environmental data and a reduction in maximal oxygen consumption.

22. The method of claim 18, wherein the estimate sufficiency model determines if the maximal oxygen consumption estimates have converged and that there are sufficient number of estimates.

23. The method of claim 18, wherein the personalized threshold model determines a confidence interval around the maximal oxygen consumption estimate based upon a population error model of maximal oxygen consumption estimates, and whether the confidence interval is below a set threshold.

24. The method of claim 18, wherein the interpretability model ensures that a final classification result is reasonable and interpretable to the user by determining if a minimum number of recent maximal oxygen consumption estimates are below the set threshold.

25. The method of claim 18, wherein a criteria evaluator is used to determine if the confidence-weighted maximal oxygen consumption estimates meet specified criteria, and if so, notifying the user that they have persistently low cardiorespiratory fitness.
